SEQUENCE LISTING (4 sequences)

SEQ ID NO: 1 - DNA, 18 bp, Artificial Sequence
  Synthetic primer
ttatctcatgcgtcatcc

SEQ ID NO: 2 - DNA, 23 bp, Artificial sequence
  Synthetic primer
gcagacaaaacaaatgaaacctc

SEQ ID NO: 3 - DNA, 20 bp, Artificial Sequence
  Synthetic primer
ttctctgagcagctccaatg

SEQ ID NO: 4 - DNA, 20 bp, Artificial sequence
  Synthetic primer
ccacagacaggccacttctt

(19) United States
(12) Patent Application Publication
(10) Pub. No.: US 2007/0042364 A1
(43) Pub. Date: Feb. 22, 2007

(54) Target genes for inflammatory bowel disease

(76) Inventors: Elizabeth E. Mannick, New Orleans, LA (US); Adriana Zapata, Metairie, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 10/540,951

(22) PCT Filed: Feb. 11, 2005
(86) PCT No.: PCT/US05/04300

§ 371 Date: Jun. 27, 2005

Related U.S. Application Data
(60) Provisional application No. 60/543,629, filed on Feb. 11, 2004.

(51) Int. Cl. C12Q 1/68

(52) U.S. Cl. 435/6

(57) ABSTRACT

Two genes have been discovered that show single nucleotide polymorphisms that are differentially expressed in patients with inflammatory bowel disease (IBD) as compared to unaffected controls. These two genes, FLJ21425 and CSF1R (colony stimulating factor 1 receptor), are located close together on chromosome 5q33 which was known to have other IBD susceptibility genes. Moreover, expression of the CSFIR gene was shown in the intestinal epithelium. These two genes can be used to test for the presence of the allele associated with IBD for an early diagnosis of susceptibility to IBD. Early identification of subjects with susceptibility to IBD will enable early treatment with known methods. Additionally, the two genes can be used to target treatment, e.g., drugs known to affect CSFIR expression. Based on gene expression data, chromosomal location and biological function, the colony stimulating factor 1 receptor gene was shown to contribute to Crohn's disease susceptibility.

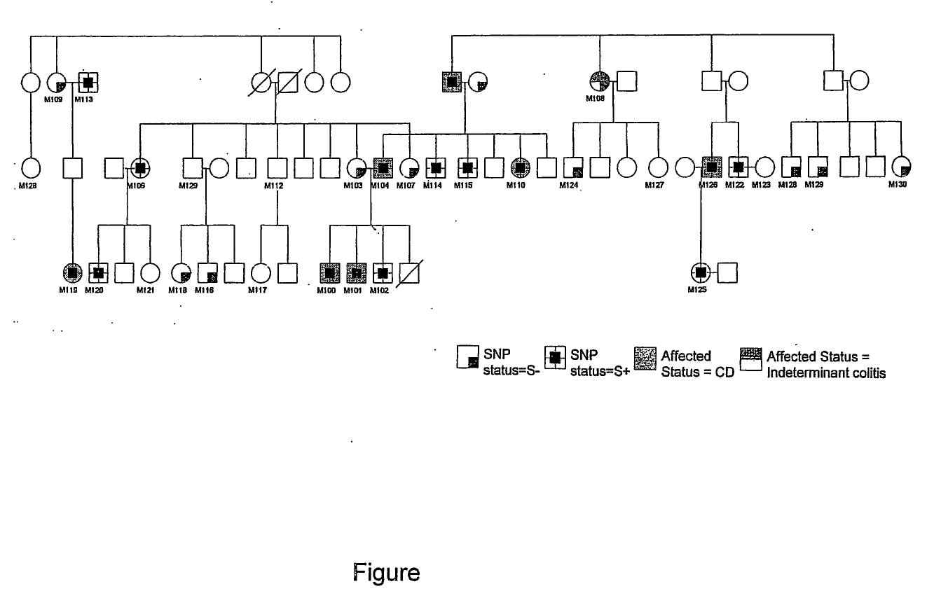

The benefit of the filing date of provisional U.S. application Ser. No. 60/543,629, filed 11 Feb. 2004, is claimed under 35 U.S.C. §119(e) in the United States, and is claimed under applicable treaties and conventions in all countries.

### TECHNICAL FIELD

This invention pertains to a method to identify patients susceptible to inflammatory bowel disease (IBD) by testing for single nucleotide polymorphisms (SNPs) in two genes, FLJ21425 and CSF1R, which were shown to be susceptibility genes for IBD, especially Crohn's disease.

### BACKGROUND ART

Crohn's disease is a chronic intestinal disorder of unknown etiology characterized by weight loss, abdominal pain, diarrhea, arthritis and the development of fistulae and abscesses. It causes significant morbidity and affects approximately 1 in 1000 individuals in the developed world. Crohn's disease is believed to ensue from the action of an environmental trigger(s) including alteration in host intestinal flora on a genetically susceptible host mucosal immune system and intestinal epithelial barrier. See F. Pallone et al., “Genetic and pathogenetic insights into inflammatory bowel disease,” Curr. Gastroenterol. Rep., vol. 5, pp. 487-92 (2003). Despite greater than fifty years of clinical experience with Crohn's disease and ulcerative colitis, collectively known as inflammatory bowel disease (IBD), the precise etiology of these diseases remains unknown and the morbidity they engender high. Further, between 10-15% of patients cannot be accurately diagnosed as having either Crohn's disease or ulcerative colitis and are classified as indeterminate colitis, making treatment decisions and evaluation of long-term prognosis difficult.

IBD susceptibility loci have been mapped to chromosomes 1, 3, 4, 6, 10, 12, 16, X and 22. See J. P. Hugot et al., “Genome-wide scanning in inflammatory bowel diseases,” Dig. Dis., vol. 16, pp. 364-369 (1998); J. Hampe et al., “A genomewide analysis provides evidence for novel linkages in inflammatory bowel disease in a large European cohort,” Am. J. Hum. Genet., vol.64, pp.808-816 (1999); and K. G. Becker et al., “Clustering of non-major histocompatibility complex susceptibility candidate loci in human autoimmune diseases,” Proc. Natl. Acad. Sci. USA, vol.95, pp. 9979-9984 (1998).

The current revolution in molecular genetics offers new hope of identifying genes that may play a role in disease susceptibility, etiology, and diagnosis. Microarray gene analysis has recently been added to the arsenal of molecular genetic techniques. See M. Schena et al., “Quantitative monitoring of gene expression patterns with a complementary DNA microarray,” Science, vol. 270, pp. 467-470 (1995). Microarray analysis allows an investigator to screen for thousands of genes in a relatively small patient sample such as a single endoscopic biopsy or a small amount of blood (<2 cc). A microarray is a glass slide, microchip, or membrane with cDNA of thousands of known sequences spotted on it. These microarrays then serve as sequence targets for hybridization to cDNA probes prepared from RNA samples from cells or tissues. A two-color fluorescence labeling technique is generally used in the preparation of the cDNA probes such that a simultaneous hybridization, but separate detection of signals, provides a comparative analysis and a determination of the relative abundance of specific genes expressed. Microarrays can be constructed from specific cDNA clones of interest, a cDNA library, or a select number of open-reading frames from a genome sequencing database to allow a large-scale functional analysis of expressed sequences. See R. A. Heller et al., “Discovery and analysis of inflammatory disease-related genes using cDNA microarrays,” Proc.Natl.Acad.Sci.USA, vol. 94, pp. 2150-2155 (1997); and M. Schena et al., “Quantitative Monitoring of Gene Expression Patterns with a Complementary DNA Microarray,” Science, vol. 270, pp. 467-470 (1995). An advantage of microarray technology is that more than one oligonucleotide sequence per gene may be included on the array, potentially providing greater specificity.

Samples from the mucosal intestinal wall of ulcerative colitis and Crohn's disease patients with inflamed and noninflamed controls have been used in a microarray analysis of approximately 6000 sequences (Affymetrix GeneChip). See B. K. Dieckgraefe et al., “Analysis of mucosal gene expression in inflammatory bowel disease by parallel oligonucleotide arrays,” Physiol. Genomics, vol. 4, pp. 1-11 (2000); I. Lawrence, C. Fiocchi, S. Chakravarti, “Ulcerative colitis and Crohn's disease: distinctive gene expression profiles and novel susceptibility candidate genes.” Hum Mol Genet 2001;10:445-56; and International Application Nos. WO 01/29269A2 and WO 02/059367. Additional attempts to diagnose inflammatory bowel disease, Crohn's disease, and ulcerative colitis by genetic differences may be found, for example, in International Application No. WO 2004/001073.

Crohn's disease (CD) and ulcerative colitis (UC), collectively known as IBD, are chronic systemic illnesses of unknown etiology with primary manifestations in the gastrointestinal tract. A major breakthrough in understanding the genetics and the pathogenesis of CD occurred with the identification of Nod2 as the first IBD susceptibility gene. This first Crohn's disease susceptibility gene, NOD2, was identified definitively by positional cloning and linkage disequilibrium mapping as well as candidate gene approaches. See Y. Ogura et al., “A frameshift mutation in NOD2 associated with susceptibility to Crohn's disease,” Nature, vol. 411, pp. 603-606 (2001); and J. P. Hugot et al., “Association of NOD2 leucine-rich repeat variants with susceptibility to Crohn's disease,” Nature, vol. 411, pp. 599-603 (2001). NOD2 encodes an intracellular receptor for muramyl dipeptide, a component of the peptidoglycan moiety of bacterial cell walls, and triggers a cascade of signaling events resulting in the activation of NF-kappa B and the host innate immune system. Crohn's disease-associated mutations in NOD2 result in defective NF kappa B activation, suggesting that Crohn's disease may represent, in part, a defect in innate immunity. See N. Inohara et al., “Host recognition of bacterial muramyl dipeptide mediated through NOD2. Implications for Crohn's disease,” J. Biol. Chem., vol. 278, pp. 5509-12 (2003). NOD2 is expressed in monocytes and in intestinal epithelial cells, including Paneth cells. See Y. Ogura et al., “Expression of NOD2 in Paneth cells: a possible link to Crohn's ileitis,” Gut, vol. 52, p.591-597 (2003)

A second gene that has recently been linked to Crohn's disease and ulcerative colitis susceptibility is the multidrug resistance transporter 1 (MDR1). A single nucleotide polymorphism (SNP) in the coding region of the gene (Ala893Ser/Thr) has been found to occur more frequently in patients with Crohn's disease and a second SNP (C3435T) has been associated with ulcerative colitis susceptibility. See S. R. Brant et al., “MDRI Ala893 polymorphism is associated with inflammatory bowel disease,” Am. J. Hum. Genet., vol. 73, pp. 1282-92 (2003); and M. Schwab et al., “Association between the C343ST MDRI gene polymorphism and susceptibility for ulcerative colitis,” Gastroenterology, vol. 124, pp. 26-30 (2003). The MDR1 gene encodes an ATP-binding cassette (ABC) family member that pumps neutral and cationic hydrophobic molecules out of the cell and plays a role in resistance to chemotherapy. Intriguingly, MDR1/-mice develop spontaneous colitis in the presence of normal intestinal bacteria. The MDR1 protein may also play a role in host defense against intracellular bacteria by extruding them from the cell, explaining its protective role in intestinal inflammation. See C. M. Panwala et al., “A novel model of inflammatory bowel disease: mice deficient fro the multiple drug resistance gene, mdr1 a, spontaneously develop colitis,” J. Immunol., vol. 161, pp. 5733-5744 (1998).

Identification of additional Crohn's disease susceptibility genes is important to complete the puzzle of Crohn's disease pathogenesis and to develop specific, targeted immunotherapies. A region of broad susceptibility to inflammatory bowel disease has been identified on chromosome 5q31-5q33 and is known as IBD5. See J. D. Rioux et al., “Genomewide search in Canadian families with inflammatory bowel disease reveals two novel susceptibility loci,” Am. J. Hum. Genet., vol. 66, pp. 1863-70 (2000). Within this region, a Crohn's disease susceptibility haplotype comprising a cytoline cluster on 5q31 has been identified. See J. D. Rioux et al., “Genetic variation in the 5g31 cytokine gene cluster confers susceptibility to Crohn disease,” Nat. Genet., vol. 29, pp. 223-8 (2001). Interestingly, a missense substitution in SLC22A4, a gene in this region that is a downstream target of the transcription factor, Runx1, is associated with susceptibility to Crohn's disease. See V. D. Peltekova et al., “Functional variants of OCTN cation transporter genes are associated with Crohn disease,” Nat. Genet., vol. 36, pp. 471-5 (2004). Moreover, an intronic SNP in a RUNX1 binding site of SLC22A4 has been found in rheumatoid arthritis, an autoimmune disease that sometime occurs in individuals and families affected by Crohn's disease. See S. Tokuhiro, “An intronic SNP in a RUNX1 binding site of SLC22A4, encoding an organic cation transporter, is associated with rheumatoid arthritis,” Nat. Genet., vol. 35, pp. 341-8 (2003). Polymorphisms in the promoter of the CD14 gene, which plays a critical role in lipopolysaccharide signaling and is located downstream from the cytokine duster, have been linked to Crohn's disease susceptibility in a case-control study. See W. Klein et al., “A polymorphism in the CD 14 gene is associated with Crohn disease,” Scand. J. Gastroenterol., vol.37, pp. 189-91 (2002).

In order to complete the IBD genetic/pathogenetic puzzle, it is critical that additional IBD susceptibility genes be identified. The Nod2 breakthrough was achieved using a positional cloning and a candidate gene approach. However, the use of genome-wide scanning and positional cloning to identify IBD susceptibility genes is costly and time consuming. Use of microarray analysis as a screening tool for the identification of candidate genes is a cost-effective and time-efficient complementary strategy.

The CSFLR (colony stimulating factor 1 receptor) gene encodes a tyrosine kinase receptor for macrophage colony stimulating factor, a cytokine that plays a significant role in monocyte differentiation. See R. Riccioni et al., “C-fms expression correlates with monocytic differentiation in PML-RAR alpha+acute promyelocytic leukemia,” Leukemia, vol. 17, pp. 98-113 (2003). It is located on chromosome 5q33, approximately 18 million base pairs from the 5q31 locus, but still within the original IBD5 locus. See J. D. Rioux et al., 2000. Although expression of CSF1R has been detected in epithelial cells of other organs, the expression of CSF1R in the intestine has not been documented. See E. Sapi et al., “Expression of CSF-1 and CSF-I receptor by normal lactating mammary epithelial cells,” J. Soc. Gynecol. Investig., vol. 5, pp. 94-101 (1998); T. Buaknecht et al., “Expression of transcripts for CSF-I and for the “macrophage” and “epithelial” isoforms of the CSF-I R transcripts in human ovarian carcinomas,” Cancer Detect. Prev., vol. 18, pp. 231-9 (1994); H. O. Smith et al., “The role of colony-stimulating factor I and its receptor in the etiopathogenesis of endometrial adenocarcinoma,” Clin. Cancer Res., vol. 1, pp. 313-25 (1995); and H. Ide et al., “Expression of colonystimulating factor I receptor during prostate development and prostate cancer progression,” Proc. Nat. Acad. Sci. USA , vol. 99, pp. 14404-9 (2002).

The CSF1R is a particularly interesting IBD candidate gene for several reasons. First, it participates in a signaling pathway in monocytes along with the G protein coupled receptor alphai2 (Gαi2) and Stat. See I. Corre et al., “Regulation by Gi2 proteins of v-fms-induced proliferation and transformation via Src-kinase and STAT3,” Oncogene, vol. 18, pp. 635-6342 (1999). Mice with a targeted disruption of either the Gαi2 or stat3 gene (in hematopoietic cells) develop spontaneous colitis, implicating this signaling pathway in the pathogenesis of IBD. See U. Rudolph et al., “Ulcerative colitis and adenocarcinoma of the colon in G alpha i2-deficient mice,” Nat. Genet., vol. 10, pp. 143-150 (1995); and T. Welte et al., “STAT3 deletion during hematopoiesis causes Crohn's disease-like pathogenesis and lethality: a critical role of STAT3 in innate immunity,” Proc. Natl. Acad. Sci. USA, vol. 100, pp. 1879-1884 (2003). Second, mutations in the CSF1R gene have been linked to myelodysplasia and acute myelogenous leukemia, two conditions with increased prevalence in patients with CD. See H. Dombret et al., “De novo acute myeloid leukemia in patients with Crohn's disease,” Nouv. Rev. Fr. Hematol., vol. 37, pp. 193-196 (1995); and G. C. Harewood et al., “Concurrent inflammatory bowel disease and myelodysplastic syndromes,” Inflamm. Bowel Dis., vol. 5, pp. 98-103 (1999). Finally, CSF1R is a target of the transcription factor, RUNX1. Polymorphisms in RUNX1 and its downstream targets are associated with genetic susceptibility to autoimmune diseases that can coexist with IBD including psoriasis, rheumatoid arthritis, systemic lupus erythematosus and type 1 diabetes. See C. Nielsen et al., “Association of a putative regulatory polymorphism in the PD-1 gene with susceptibility to type 1 diabetes,” Tissue Antigens, vol. 62, pp. 492-497 (2003); C. Helms et al., “A putative RUNX1 binding site variant between SLC9A3R1 and NAT9 is associated with susceptibility to psoriasis,” Nat. Genet., vol. 35, pp. 349-356 (2003); S. Tokuhiro et al., “An intronic SNP in a RUNX1 binding site of SLC22A4, encoding an organic cation transporter, is associated with rheumatoid arthritis,” Nat. Genet., vol. 35, pp. 341-348 (2003); and L. Prokunina et al., “A regulatory polymorphism in PDCD1 is associated with susceptibility to systemic lupus erythematosus in humans,” Nat. Genet., vol. 32, pp.666-669 (2002).

The CSF1R gene has been shown to be overexpressed in a variety of pathologic conditions including cancer. See B. M. Kacinski, “CSF-1 and its receptor in breast carcinomas and neoplasms of the female reproductive tract,” Mol. Reprod. Dev., vol. 46, pp. 71-74 (1997). A number of therapeutic agents such as corticosteroids, retinoic acids, interleukin 10, and vitamin D3 been shown to work in part by either increasing the expression of CSF1 and/or altering the expression of CSF1R. See E. Sapi et al., “Transcriptional regulation of the c-fins (CSF-1R) proto-oncogene in human breast carcinoma cells by glucocorticoids,” Oncogene, vol. 10, pp. 529-42 (1995); K. Zhu et al., “Vitamin D(3) and analogues modulate the expression of CSF-1 and its receptor in human dendritic cells.,” Biochem. Biophys. Res. Commun., vol. 297, pp. 1211-1217 (2002); E. Sapi et al., “Effect of all-trans-retinoic acid on c-fins proto-oncogene [colony-stimulating factor 1 (CSF-1) receptor] expression and CSF-1-induced invasion and anchorage-independent growth of human breast carcinoma cells,” Cancer Research, vol. 59, pp. 5578-5585 (1999); and C. Rieser et al., “Human monocyte-derived dendritic cells produce macrophage colony-stimulating factor: enhancement of c-fms expression by interleukin-10,” Eur. J. Immunol., vol. 28, pp. 2283-88 (1998). Mifepristone (RU-486) has been shown to inhibit the glucocorticoid-induced increase in CSF-1R expression in breast carcinomas. See B. M. Kacinsli et al., “RU-486 can abolish glucocorticoid-induced increases in CSF-1 receptor expression in primary breast carcinoma specimens,” J. Soc. Gynecol. Investig., vol. 8, pp. 114-116 (2001).

### DISCLOSURE OF INVENTION

We have discovered two genes that show single nucleotide polymorphisms that are differentially expressed in patients with inflammatory bowel disease (IBD) as compared to unaffected controls. These two genes, FLJ21425 and CSF1R (colony stimulating factor 1 receptor), are located close together on chromosome 5q33 which was known to have other IBD susceptibility genes. Moreover, expression of the CSF1R gene was shown in the intestinal epithelium. These two genes can be used to test for the presence of the allele associated with IBD for an early diagnosis of susceptibility to IBD. Early identification of subjects with susceptibility to IBD will enable early treatment with known methods. Additionally, the two genes can be used to target treatment, e.g., drugs known to affect CSF1R expression. Based on gene expression data, chromosomal location and biological function, the colony stimulating factor 1 receptor gene was shown to contribute to Crohn's disease susceptibility.

### BRIEF DESCRIPTION OF DRAWING

The FIGURE illustrates the pedigree of an Acadian family that is affected by Cohn's disease (CD), showing the presence of CD and/or the single nucleotide polymorphism of the FLJ21425 gene for individuals tested.

### MODES FOR CARRYING OUT THE INVENTION

### EXAMPLE 1

Materials and Methods

Patients. Patients (n=111) and controls (n=108) were recruited in the study from Children's Hospital of New Orleans and private practices in Southeastern Louisiana and Western Mississippi after Louisiana Health Sciences Center Institutional Review Board (IRB) approval and informed consent and assent. Patient and control DNA were also obtained from archival colonic tissue blocks after IRB approval.

DNA Extraction and Purification. Genomic DNA was obtained from one of three sources for all subjects: peripheral blood buffy coat, buccal swab, or paraffin-embedded archival tissue blocks. For blood, ten ml of whole blood was collected in purple top, EDTA tubes; and buffy coats prepared using red blood cell lysis buffer (NH₄Cl, NH₄HC0₃, H₂0), pellet buffer (1 M Tris HCl pH 8.0, 0.5 M EDTA, NaCl, H₂0), 10% SDS and Proteinase K. Buffy coats were heated in a water bath overnight at 56° C. and stored at −20° C. DNA was extracted using phenol:chloroform:isoamyl alcohol, followed by chloroform, and precipitated in 100% ethanol. After air drying, the pellet was resuspended in TE buffer and its quantity and integrity were verified by 1% agarose gel and spectrophotometry (Beckman Coulter, DU640B).

DNA was extracted from buccal swabs (EpicentreTechnologies, Madison, Wis.) following the manufacturer's instructions. Briefly, swabs were placed in DNA extraction solution, mixed for ten seconds, and incubated at 60° C. for 30 min, then a total of 16 min at 98° C. After centrifugation at 10,000×g at 4° C., the supernatant was transferred to a dean tube and stored at −20 ° C.

For archival tissue blocks, 3 sections of 10 μm were cut and incubated twice with 1 ml of n-octane (Sigma, St. Louis, Mo.) at 56° C. for 15 min. After centrifugation at 10,000×g at room temperature (RT), the pellet was resuspended in 1 ml 100% EtOH and then in 1 ml 75% EtOH. After the last centrifugation, the pellet was resuspended in 85 μl of pellet buffer (10 mM Tris-HCL, ph 8.0, 10 mM EDTA, pH 8.0, 150 mM NaCl) followed by 5 μl of Proteinase K (20 mg/ml) (Invitrogen, Grand Island, N.Y.) and 10 μl 10% SDS (Invitrogen). The samples were incubated overnight at 56° C. One hundred μl of phenol chloroform:iso-amylalcohol (50:1) (Sigma) was added and the sample was centrifuged at 10,000×g for 5 min at RT. The aqueous phase was transferred to a clean tube and 100 ul of chloroform were added (Sigma). The sample was centrifuged at 10,000×g for 5 min and the aqueous phase transferred to a clean tube and mixed with 200 μl 100% ethanol (Aldrich) and incubated at −70° C. for at least 1 hr. The DNA was precipitated by centrifugation and resuspended in TE buffer. The DNA concentration was determined by UV spectrophotometry.

Polymerase Chain Reaction (PCR). Forward and reverse primers to amplify DNA in the vicinity of the single nucleotide polymorphism (SNP) of interest were designed using the Primer Quest^{SM} (Integrated DNA Technologies (IDT), Coralville, Iowa) program and ordered from IDT. For example, the primer sequences for CSF1R are: (F) 5′TTC TCT GAG CAG CTC CAA TG3′ (SEQ. ID. No. 3) and (R) 3′CCA CAG ACA GGC CAC TTC TT5′ (SEQ. ID. No. 4). Master Mix for PCR was prepared using Taq polymerase, dNTPs and other reagents from Invitrogen (Carlsbad, Calif.). After optimization of conditions, PCR reactions were carried out in a Bio-Rad I-cycler. The PCR product was resolved on a 1% agarose gel and purified using Qiaquick DNA Purification Kit (Qiagen, Inc.; Valencia, Calif.).

DNA Sequencing. Forward and Reverse DNA sequencing was performed in the Louisiana State University Gene Sequencing Core. Briefly, in a 0.2 mL PCR tube, DNA template, primer, BigDye Terminator Ready Reaction Mix (PE Applied Biosystems, Foster City, Calif.), 5× sequencing mix, and HPLC water were combined to 20 μl. Tubes were then placed in a thermal cycler (GeneAmp PCR 9700) set to the following program.

| | |
|---|---|
| 30 cycles | 96° C. - 10 seconds |
| | 58° C. - 5 seconds |
| | 60° C. - 4 minutes |

Extension products were purified by adding 3M NaOAc, pH 4.6 and 95% EtOH to reaction tubes for 20 min, and spinning tubes upright at 3600 rpm for 30 min. Tubes were then inverted and spun at 700 rpm for 1 min. After washing the pellet in 70% EtOH, tubes were spun at 3600 for 10 min. The procedure beginning with inversion of tubes was repeated, and tubes centrifuged at 700 rpm for 1 min and air dried. To analyze the sequencing reaction, formamide was added to each tube. Denaturation lasted for 3 min at 95° C., followed by immersion in wet ice. The sequencing gel was prepared using urea, HPLC water, Long Ranger 50% (PE Applied Biosystems, Weiderstadt, Germany), and 10× TBE buffer, stirring for 1 h. 10% ammonium persulphate (APS) and TEMED was added to the filtered gel solutions, and gel was loaded into a cassette with glass plates in an ABI 3100 automated sequencer equipped with ABI PRISM Data Collection Software. 1× TBE was used as running buffer for gel electrophoresis. Fluorescent dye labels were used to incorporate into DNA extension products. Four different dyes were used to identify the A, C, G, and T extension reactions using an argon laser.

Immunohistochemistry. Slides cut from paraffin-embedded tissue blocks were deparaffinized, hydrated, and blocked with 3% hydrogen peroxide at room temperature for 15 min. After rinsing in distilled water, they were placed in PBS for 2 min and then blocked with Biocare's Background Sniper (Biocare Medical, Walnut Creek, Calif.) for 10 min at RT. Slides were incubated with primary antibody (rabbit polyclonal antibody to human c-fms, Cymbus Biotechnology, Ltd., Chandlers Ford, Hants, United Kingdom) at a dilution of 1:100 for 60 min at RT and, after rinsing with PBS, incubated with secondary antibody (MACH 2 Rabbit-HRP Polymer, Biocare Medical) for 30 min at RT. After rinsing with PBS, slides were placed in diaminobenzamide for 7 min at RT, rinsed in 2 changes of distilled water, counterstained with hematoxylin, dehydrated and mounted with resinous medium.

Data Analysis: Numbers of patients with a given allele of a SNP were compared to numbers of controls using a chi-square statistic or a Fischer's exact test. An odds ratio with 95% confidence interval was calculated for disease association for each SNP using SAS software (SAS, Cary, N.C.).

### EXAMPLE 2

Microarray Identification of Differentially Expressed Genes in CD Patients

Data have been gathered from microarray studies of gene expression in peripheral blood mononuclear cells (PBMCs) and colonic tissue biopsies from IBD patients, using methods as described in International Application WO 02/059367. The first study used a 2400 gene array to analyze mRNA from PBMCs from patients with CD, patients with UC and patients with other gastrointestinal inflammatory conditions (MICROMAX, PerkinElmer Life Sciences, Boston, Mass.). Briefly, this study identified several novel categories of differentially expressed genes in CD, including genes related to differentiation and leukemogenesis, signal transduction in lipid rafts and vesicular trafficking. (Data not shown) The second study examined mRNA expression from PBMCs and colonic biopsies from 6 patients with newly diagnosed, untreated CD, 4 patients with newly diagnosed UC, and 8 controls using a 35,000 gene glass slide array (Incyte Genomics, Palo Alto, Calif.). Many inflammation-related genes were over-expressed. Of the 6 underexpressed genes, 2 (FLJ21425 and SLC26A2) were located on chromosome 5q33 adjacent to one another and in close proximity to a third, overexpressed gene (CSF1R). These three genes were then sequenced in patients with CD and controls.

From the second microarray study, only 5 genes were significantly underexpressed in the colons of patients with active, untreated Crohn's disease (CD). Of these 5 genes, two were neighboring genes, FLJ21425 and SLC26A2 (solute carrier family 26, member A2), on chromosome 5q33. FLJ21425 is a hypothetical gene with little homology to any known gene. A search of the InterPro database, however, indicated some homology with ubiquitin E3 ligases from a number of invertebrate species. SLC26A2 is a sulfate taansporter. Mutations in this gene are known to cause diastrophic dysplasia, a form of dwarfism. A significantly overexpressed gene, CSF1R, was located within 60 kB of these two genes. These three genes are located on chromosome 5q33 in a locus that has been linked to IBD susceptibility. See Rioux et al., 2000.

### EXAMPLE 3

Prevalence of SNP of FLJ21425 in CD Patients

The initial hypothesis was that underexpression of the two contiguous genes, FLU21425 and SLC26A2, might be due to a microdeletion involving both genes, especially because both somatic and germline chromosome 5q deletions have been reported in a variety of syndromes, including myelodysplasia. In sequencing FLJ21425 and SLC26A2 cDNA, however, no deletion was found. However, a single nucleotide polymorphism (SNP), designated C2473T, was found in 50% (n=6) of the CD patients, but in only 12.5% (n=8) of controls. In 2 of the 3 CD patients with the T allele of the SNP, cDNA expression was decreased by more than two-fold in the microarray data. Therefore, the C2473T polymorphism in the FLJ21425 gene could be a risk factor for Crohn's disease.

To test this hypothesis, genomic DNA was sequenced from 60 patients with CD and 100 controls. The T allele (CT or TT) of the SNP was present in 42% of unrelated patients with CD and 27% of controls (Table 1). When data from affected and unaffected family members in several large, Acadian pedigrees was analyzed, the T allele of the SNP cosegregated with the disease. One example is shown in the Figure. The figure indicates the pedigree of an Acadian family that had several members affected by CD and indeterminate colitis. The shaded squares and circles indicate individuals with CD. Squares with crosses represent individuals with a CT or a TT allele of the C2473T polymorphism. Shading in the lower right hand quadrant of a square or circle indicates individuals who have the CC (wild-type) allele. Blank squares and circles are individuals who have not been tested. However, unaffected family members in some pedigrees tested had the T allele of the SNP. This suggests that they may have increased susceptibility to the disease. Alternatively, the T allele of the SNP in FLJ21425 could be in linkage disequilibrium with another disease-associated gene such as CSF1R or one of several candidate genes in the 5q31 cytokine cluster. See J. D. Rioux et al., “Genetic variation in the 5q31 cytokine cluster confers susceptibility to Crohn disease,” Nat. Genet., vol. 29, pp. 223-8 (2001).

TABLE 1
Crohn's Disease Status and FLJ21425 SNP in Unrelated Patients

| | CD− | | CD+ | | Totals |
|---|---|---|---|---|---|
| SNP − (CC genotype; wild-type) | 72 | (0.73) | 28 | (0.58) | 100 |
| SNP + (CT or TT genotype) | 27 | (0.27) | 20 | (0.42) | 47 |
| Totals | 99 | | 48 | | 147 |

χ² = 3.08 (p < 0.10)

### EXAMPLE 4

Prevalence of SNP of CSF1R in CD Patients

The colony stimulating factor receptor (CSF1R/c-fms) gene encodes a tyrosine kinase receptor for macrophage colony stimulating factor, a cytokine that plays an important role in monocyte differentiation. See R. Riccioni et al., “C-fms expression correlates with monocytic differentiation in PML-RAR alpha+acute promyelocytic leukemia,” Leukemia, vol. 17, pp. 98-113 (2003). Because CSF1R is located within 60 kB of FLJ21425 and because it was overexpressed in colons of patients with CD, polymorphisms in CSF1R were examined in IBD patients. Particular areas of interest were the promoter regions of the gene and the fms intronic regulatory element (FIRE) regions of the gene since these are known to contain RUNX1 binding sites. The two promoter regions and the FIRE regions of the gene have been partially sequenced using genomic DNA from patients and controls. The results are shown in Table 2. As shown, patients with CD have a higher frequency than controls of the minor alleles of several SNPs in this region. For one of these SNPs, an A-T polymorphism close to a RUNX1 binding site in the second intron of the gene, the difference between CD patients and controls was highly significant, with 40% of the CD patients carrying the T allele and only 7% of controls.

TABLE 2
Prevalence of SNPs in Promoter and c-FMS Intronic Regulatory
Elements (FIRE) in Unrelated Patients with CD vs Controls

| | Promoter | FIRE 1 | FIRE 1 |
|---|---|---|---|
| | 1:2088 | (Intron 1):2012 | (Intron 1):2033 |
| SNPs | G→C | C→A | A→T |
| CD | 3/41 | 2/40 | 16/40 |
| | (0.073) | (0.05) | (0.40) |
| CONTROL | 1/67 | 0/42 | 3/42 |
| | (0.015) | (0.00) | (0.07) |
| p-value (χ²) | <0.20 | <0.20 | <0.001 |
| | (2.5) | (2.15) | (12.42) |

Based on these results, CSF1R and/or FLJ21425 are IBD susceptibility genes. As of yet, a genotype/phenotype association has not been detected in CD patients who have the CT or TT allele of the FLJ21425 SNP or the AT or TT allele of the CSF1R SNP. Preliminary data involving small numbers of patients suggested that Acadians (“Cajuns” of French Canadian descent) with CD have a disproportionate rate of the T allele of the C2473T SNP of FLJ21425 and of the T allele of the A2033T SNP of CSF1R. Of 9 Acadian CD patients tested for the FLJ21425 SNP, 6 (66%) have a T allele. Of 9 Acadian CD patients tested for the CSF1R SNP, 6 also (66%) have a T allele.

### EXAMPLE 5

Association of the T allele of the A2033T SNP with Crohn's Disease Especially Inpatients of Acadian Descent

A SNP (A2033T; this SNP occurs 2033 base pairs from the 3′ end of the eleventh exon of the CSF1R gene) was detected in the eleventh intron of the CSF1R gene that was located 77 base pairs downstream from a RUNX1 binding site (TGTGGT). Forward and reverse sequencing of this SNP was performed in 111 patients with Crohn's disease and 108 controls (Table 3). Thirty patients with Crohn's disease (27%) but only fourteen controls (13%) had the T allele of the SNP (χ²=6.74, p<0.01, O.R.=2.49 with 95% confidence interval, 1.23<O.R.<5.01).

TABLE 3
Crohn's Disease Status vs CSF I R A2033T SNP: All Patients

| | | |
|---|---|---|
| CD− | CD+ | Total |

| | | | | | |
|---|---|---|---|---|---|
| T Allele Absent | 94 | (87%) | 81 | (73%) | 175 |
| T Allele Present | 14 | (13%) | 30 | (27%) | 44 |
| Total | 108 | | 111 | | 219 |

χ² = 6.74 (p < 0.01)
O.R.: 249 (1.23 < O.R. < 5.01)

Data from the stratification of Crohn's patients and control patients by ethnicity (Table 4) suggested that the differential expression of the SNP was more pronounced in some ethnic groups (Acadian) than in others (African American). In the case of Crohn's patients of Acadian descent, the rate of the T allele (47% vs. 23%) was significantly higher than the rate of the T allele in all other ethnicities combined (Table 5; χ²=4.01, p<0.05, O.R.=3.04 with 95% confidence interval, 1.09<O.R.<8.47).

TABLE 4
A2033T SNP Allele by Ethnicity

| | T Allele Absent | | T Allele Present | | Total |
|---|---|---|---|---|---|
| CROHN'S | | | | | |
| PATIENTS | | | | | |
| Acadian | 10 | (53%) | 9 | (47%) | 19 |
| African-American | 17 | (85%) | 3 | (15%) | 20 |
| Caucasian | 44 | (75%) | 15 | (25%) | 59 |
| Hispanic | 2 | (100%) | 0 | (0%) | 2 |
| Jewish | 4 | (80%) | 1 | (20%) | 5 |
| Unknown | 4 | (67%) | 2 | (23%) | 6 |
| Total | 81 | | 30 | | 111 |
| CONTROL | | | | | |
| PATIENTS | | | | | |
| Acadian | 36 | (84%) | 7 | (16%) | 43 |
| African-American | 4 | (57%) | 3 | (43%) | 7 |
| Caucasian | 32 | (91%) | 3 | (9%) | 35 |
| Hispanic | 7 | (87%) | 1 | (13%) | 8 |
| Jewish | 3 | (100%) | 0 | (0%) | 3 |
| Unknown | 12 | (100%) | 0 | (0%) | 12 |
| Total | 94 | | 14 | | 108 |

TABLE 5
Ethnicity vs CSF I R A2003T SNP: Crohn's Patients

| | | |
|---|---|---|
| Acadian | Non-Acadian | Total |

| | | | | | |
|---|---|---|---|---|---|
| T Allele Absent | 10 | (53%) | 71 | (77%) | 81 |
| T Allele Present | 9 | (47%) | 21 | (23%) | 30 |
| Total | 19 | | 92 | | 111 |

χ² = 4.81 (p < 0.05)
O.R.: 3.04 (1.09 < O.R. < 8.47)

Since there were more controls of Acadian descent than patients of non-Acadian descent (Table 4), it is unlikely that the higher rate of the T allele of the A2033T SNP noted in Crohn's patients in general (Table 3) can be attributed to ethnicity. However, to exclude the possibility that Acadian ethnicity was a confounding variable, we compared rates of the T allele in non-Acadian patients with Crohn's disease to those of non-Acadian controls (Table 6). In this analysis, patients with Crohn's disease still had significantly higher rates of the T allele than controls.

TABLE 6
Crohn s Disease Status vs CSF I R
A2033T SNP: Non-Acadian Patients

| | | |
|---|---|---|
| CD | CD+ | Total |

| | | | | | |
|---|---|---|---|---|---|
| T Allele Absent | 58 | (89%) | 71 | (77%) | 129 |
| T Allele Present | 7 | (11%) | 21 | (23%) | 28 |
| Total | 65 | | 92 | | 157 |

Fisher exact test (p = 0.025)
O.R.: 2.45 (0.97 < 6.17)

### EXAMPLE 6

The CSF1R Protein is Expressed in the Superficial Epithelium of the Ileum and Colon

Paraffin-embedded, formalin-fixed tissue sections from control patients were stained with a rabbit anti-human polyclonal antibody to CSF1R and read by a pathologist. Negative controls were stained with secondary antibody alone. Positive cytoplasmic staining was noted in the superficial epithelium of the ileum and colon with differentiated cells being sloughed off into intestinal lumen staining most vividly (Data not shown). Examination of the intracellular staining pattern revealed a characteristic staining pattern with the terminal web and the lateral junctions of intestinal epithelial cells.

The expression of the CSF1R protein was found to located in the cytoplasm of certain epithelial cells of the superficial epithelium and villous tips of the ileum and colon, including cells that were being sloughed into the lumen. Because of this superficial location of staining, CSF1R protein may play a role in differentiation of intestinal epithelial cells as it does in macrophages. The most intense cytoplasmic staining occurred in the terminal web of the epithelial cell and in the lateral junctions of the cells.

### EXAMPLE 7

Further Confrontation of Susceptibility Genes

To further confirm that either FLJ21425 or CSF1R, genes located close together on chromosome 5q33 and previously identified by microarray analysis, is an IBD susceptibility gene, further sequencing and linkage analysis will be performed.

As shown above in Example 4, a minor allele of an SNP in CSF1R was found in 40% of patients with CD and in 7% of controls. Additionally, as shown in Example 3, a minor allele of an SNP in FLJ21425 was found present in 45% of patients with CD and in 27% of healthy controls. Furthermore, the presence of the T allele of the FLJ21425 SNP cosegregated with disease in several large, multiply affected pedigrees. However, the allele was also present in some unaffected family members. These results suggested incomplete penetrance, meaning that other genetic factors (possibly in linkage disequilibrium with the SNP) and environmental factors contribute to the differential prevalence of the SNP in patients with CD.

First, the above CSF1R and FLJ21425 findings in CD will be further confirmed and extended to patients with ulcerative colitis (UC) by recruiting at least 125 subjects per disease group and examining their DNA for the CSF1R A2033T SNP and the FLJ21425 C2473T SNP. Allele-specific PCR will be used to confirm the results for FLJ21425 and to test for the presence of the FLJ21425 SNP in newly recruited patients. See S. S. Sommer et al., “PCR amplification of specific alleles (PASA) is a general method for rapidly detecting known single-base changes,” Biotechniques, vol. 12, pp. 82-87 (1992); and J. P. Struewing et al., “The risk of cancer associated with specific mutations of BRCA1 and BRCA2 among Ashkenazi Jews,” N. Engl. J. Med., vol. 336, pp. 1401-1408 (1997). A variety of databases have been searched looking for restriction enzyme sites within the immediate vicinity of the FLJ21425 SNP to no avail. To detect and/or confirm the presence or absence of the CSF1R SNP, PCR will be used with restriction enzyme digestion of the SNP site. (A PvuII site that contains the SNP has been found).

Next, the possibility that other genes in the vicinity of the SNPs identified above are in linkage disequilibrium with the SNPs and may be contributing to the differential prevalence of the minor alleles of the SNPs in CD will be investigated. Microsatellite markers and SNPs in the vicinity of FLJ21425 and CSF1R will be examined, and linkage analysis in a larger number of multiplex families with more than one family member affected by IBD will be performed. At least 25 multiplex, Acadian families will be analyzed (currently 12 families with at least 2 affected individuals have been examined, including the one shown in the Figure). The Acadian population is particularly interesting since, based on very small numbers, they appear to be over represented among CD patients with the SNPs and because they share a common ancestry.

Allele-specific polymerase-chain-reaction (PCR): Two PCR reactions will be performed on each sample, one with each forward primer and a common reverse primer. Primers will be designed such that the polymorphic nucleotides (in the case of the FLJ21425 SNP of interest, C and T, and in the case of the CSF1R SNP, A and T) are the 3′ nucleotide of each forward primer using the Primer Quest package (IDT). PCR will be performed as described above, as modified below. In each 96-well plate, DNA from individuals known to be heterozygotes and homozygotes will be run as controls.

Restriction enzyme digestion and PCR. DNA amplified with primers flanking the CSF1R SNP (see below) will be digested using the PvuII restriction enzyme, which requires the following sequence for cutting (polymorphic nucleotide shown in bold): CAGCTG. After digesting samples, DNA will be run on a 10% acrylamide gel. Using this enzyme, DNA with wild-type (A) alleles will be cut into a 200 and 250 bp fragment, while DNA from patients with the T allele will not be cut into these two fragments.

Primer sequences. Before sequencing or performing restriction enzyme digestion or allele-specific PCR, approximately 500 bp of DNA containing the SNP of interest will be amplified using primers that have been used successfully to prepare DNA for sequencing.

| | |
|---|---|
| C2473T SNP (FLJ21425): | |
| Forward primer- | |
| 5′-TTATCTCATGCGTCATCC-3′ | (SEQ. ID. No. 1) |
| Reverse primer- | |
| 3′-GCAGACAAAACAAATGAAAGCTG-5′ | (SEQ. ID. No. 2) |
| A2033T SNP (CSF1R): | |
| Forward primer- | |
| 5′-TTCTCTGAGCAGCTCCAATG-3′ | (SEQ. ID. No. 3) |
| Reverse primer- | |
| 3′-CCACAGACAGGCCACTTCTT-5′ | (SEQ. ID. No. 4) |

The thermocycler is set to the following programs:

(FLJ21425)

3 cycles 96° C.—3 min
94° C.—40 sec
60° C.—40 sec
72° C.—1 min

32 cycles 94° C.—40 sec
58° C.—30 sec
72° C.—1 min
72° C.—10 min

(CSF1R)

3 cycles 96 ° C.—3 min
94° C.—40 sec
60 ° C.—40 sec
72 ° C.—1 min

32 cycles 94° C.—40 sec
63° C.—30 sec
72° C.—1 min
72° C.—10 min

The forward primers are also used for sequencing.

Linkage analysis. Initially genotype microsatellite markers from a commercially available ABI panel for chromosome 5 in the vicinity of FLJ21425 and CSF1R on chromosome 5q33 will be genotyped. If linkage analyses (see below) of these markers in the Acadian families are not consistent with the IBD locus being in this region, genotyping will be extended to the ABI marker panel of approximately 400 polymorphic microsatellite markers (average spacing of 10 centiMorgans). Allele frequencies for these markers in the Acadian population have been previously estimated.

PCR amplification will be carried out according to protocols provided by ABI using a Perkin Elmer thermal cycler. The amplified samples will be genotyped using an ABI 3100 automated sequencer and the ABI genotyper software, and the marker genotype data will be checked for Mendelian inconsistencies using the program GCONVERT.

Parametric (model-based) linkage analysis of the phenotype and genotype data for the families will be performed using the program LODLINK from the S.A.G.E package. A dominant model with incomplete penetrance (and possibly others) will be used for the IBD locus, and the maximum lod score will be adjusted for multiple testing, if necessary. See S. E. Hodge, “Model-free vs. model-based linkage analysis: a false dichotomy?”, Am. J. Med. Genet., vol. 105, pp. 62-64 (2001). Further analysis will be performed with additional markers within regions that give two-point and multi-point lod scores of greater than 1.5.

Data Analysis: Statistical association between disease status and the C2473T SNP of FLJ21425 or the A2033T SNP of CSF1R will be evaluated by chi-square analysis as described above in Example 1.

The statistical association between the C2473T polymorphism in FLJ21425 or the A2033T SNP in CSF1R and CD and/or UC is expected to be confirmed. If an association is found with the CSF1R SNP, the entire gene will be sequenced in patients versus controls to look for other polymorphisms that might be disease-associated. The cell-specific expression of CSF1R in colonic tissue and PBMC's from IBD patients with and without the T allele will be examined. If an over expression of CSF1R in CD and/or UC is found, the phenotype of experimental colitis in CSF1R transgenic mice and/or knock-out mice will be examined.

If an association of IBD with FLJ21425 is confirmed, cell culture studies will be conducted to characterize the protein products of this gene. These studies will involve cloning the gene, over expressing the gene with an adenoviral vector and under expressing the gene with siRNA. In addition, experiments to determine if expression of FLJ21425 is altered in patients with the T allele of the FLJ21425 SNP will be performed as suggested by preliminary data.

The complete disclosures of all references cited in this application are hereby incorporated by reference. Also incorporated by reference is the complete disclosure of the following document: A. Zapata-Velandia et al., “Association of the T allele of an intronic single nucleotide polymorphism in the colony stimulating factor 1 receptor with Crohn's disease: a case-control study,” Journal of Immune Based Therapies and Vaccines, vol. 2, pp. 6-14 (2004). In the event of an otherwise irreconcilable conflict, the present specification shall control.

What is claimed:

1. A method for diagnosing a genetic susceptibility for an inflammatory bowel disease in a subject, such method comprising:
a. obtaining a biological sample containing nucleic acid from said subject, and
b. analyzing said nucleic acid to detect the presence or absence of a single nucleotide polymorphism in a gene selected from the group consisting of FLJ21425 and CSF1R, wherein said single nucleotide polymorphism is associated with a genetic predisposition for inflammatory bowel disease.

2. The method of claim 1, wherein at least one single nucleotide polymorphism is located at 2033 base pairs from the 3′ end of the eleventh intron of the CSF1R gene.

3. The method of claim 1, wherein said nucleic acid is DNA, RNA, cDNA or mRNA.

4. The method of claim 1, wherein said analysis is accomplished by sequencing, mini sequencing, hybridization, restriction fraction analysis, oligonucleotide ligation assay or allele specific PCR.

5. The method of claim 4, wherein said analysis is accomplished using primers selected from the group consisting of SEQ. ID. No. 1, SEQ. ID. No. 2, SEQ. ID. No. 3, and SEQ. ID. No. 4.

6. A method as in claim 1, wherein said inflammatory bowel disease is Crohn's disease.

7. A method of treatment or prophylaxis in a subject, said method comprising:
a. obtaining a sample of biological material containing nucleic acid from a subject;
b. analyzing said nucleic acid to detect the presence or absence of a least one single nucleotide polymorphism of a gene selected from the group consisting of FLJ21425 and CSF1R associated with a genetic predisposition for inflammatory bowel disease; and
c. treating the subject for inflammatory bowel disease.

8. The method of claim 7, wherein at least one single nucleotide polymorphism is located at 2033 base pairs from the 3′ end of the eleventh intron of the CSF1R gene.

9. The method of claim 7, wherein said nucleic acid is DNA, RNA, cDNA or mRNA.

10. The method of claim 7, wherein said analysis is accomplished by sequencing, mini sequencing, hybridization, restriction fraction analysis, oligonucleotide ligation assay or allele specific PCR.

11. The method of claim 10, wherein said analysis is accomplished using primers selected from the group consisting of SEQ. ID. No. 1, SEQ. ID. No. 2, SEQ. ID. No. 3, and SEQ. ID. No. 4.

12. A method as in claim 7, wherein said inflammatory bowel disease is Crohn's disease.

13. A method as in claim 7, wherein said treatment comprises administering to the subject one or more drugs known to affect expression of the CSF1R gene.

14. A method as in claim 13, wherein said drugs are selected from the group comprising corticosteroids, retinoic acids, interleukin 10, vitamin D3, and mifepristone.
